(19) United States
(12) Patent Application Publication
(10) Pub. No.: US 2025/0281539 A1
(43) Pub. Date: Sep. 11, 2025

(54) METHODS OF CULTURING TUMOR INFILTRATING LYMPHOCYTES

(71) Applicant: H. LEE MOFFITT CANCER CENTER AND RESEARCH INSTITUTE, INC., Tampa, FL (US)

(72) Inventors: Shari PILON-THOMAS, Land O Lakes, FL (US); Nina OBERTOPP, Tampa, FL (US); Matthew BEATTY, Riverview, FL (US); Jad CHAHOUD, Tampa, FL (US); Mohammed A. ALKHOULI, Wesley Chapel, FL (US)

(21) Appl. No.: 18/863,838

(22) PCT Filed: May 4, 2023
(86) PCT No.: PCT/US2023/021044

§ 371 Date: Nov. 7, 2024

Related U.S. Application Data
(60) Provisional application No. 63/340,155, filed on May 10, 2022.
(60) Provisional application No. 63/340,157, filed on May 10, 2022.

(51) Int. Cl. A61K 35/17; A01K 67/027; A61K 40/11; A61P 35/00; C12N 5/00; C12N 5/0783

(52) U.S. Cl. CPC A61K 35/17; A01K 67/027; A61K 40/11; A61P 35/00; C12N 5/0018; C12N 5/0638; A01K 2227/105; A01K 2267/0331; C12N 2500/02; C12N 2501/2302; C12N 2501/51; C12N 2501/515; C12N 2501/52; C12N 2502/00

(57) ABSTRACT

Disclosed are methods for expanding tumor infiltrating lymphocytes (TILs) and driving TILs towards a memory T cell phenotype. Also disclosed is the creation of a mouse model to investigate adoptive cell therapy using TILs.

### CROSS-REFERENCE TO RELATED APPLICATIONS

This application claims the benefit of U.S. Provisional Application No. 63/340,155, filed on May 10, 2022, and U.S. Provisional Application No. 63/340,157, filed on May 10, 2022, applications which are incorporated herein by reference in their entirety.

### I. Government Support

This invention was made with government support under Grant No. CA244100 awarded by NIH. The government has certain rights in the invention.

### II. BACKGROUND

Recent advances in immunotherapy checkpoint inhibitors and adoptive cell therapy (ACT) using tumor-infiltrating lymphocytes (TIL) has greatly improved treatment options for cancer patients. The success of ACT with TIL is highly dependent on the expansion of high numbers of tumor-reactive TIL. However, many patients develop resistance to these therapies and the overall prognosis remains poor. What are needed are new models and strategies for improving TIL therapy.

### III. SUMMARY

Disclosed are methods and compositions related to expanding tumor infiltrating lymphocytes and the creation of a mouse model for studying TILs used in adoptive cell therapy (ACT).

In one aspect, disclosed herein are methods of expanding tumor infiltrating lymphocytes (TILs) and/or increasing the number of Trm in a TIL population comprising a) obtaining a population of TILs (such as, for example, primary TILs obtained from an autologous, allogeneic, or syngeneic donor source); b) culturing the TILs in a first pre-rapid expansion protocol (pre-REP) culture comprising media comprising IL-2 and incubating the TILs in a normoxic oxygen environment (i.e., 20-22% oxygen); c) culturing the TILs in a second pre-REP culture comprising IL-2 and incubating the TILs in a normoxic (i.e., 20-22% oxygen) or hypoxic oxygen environment (i.e., 0-19.99% oxygen); and d) expanding TILs using a rapid expansion protocol (REP) in the presence of anti-CD3 antibody, IL-2, and allogeneic feeder cells incubating the TILs in a normoxic (i.e., 20-22% oxygen) or hypoxic (i.e., 0-19.99% oxygen) oxygen environment.

Also disclosed herein are methods of expanding tumor infiltrating lymphocytes (TILs) and/or increasing the number of Trm in a TIL population of any preceding aspect, wherein the TILs are grown in a normoxic oxygen environment (i.e., 20-22% oxygen) in the second pre-REP culture.

In one aspect, disclosed herein are methods of expanding tumor infiltrating lymphocytes (TILs) and/or increasing the number of Trm in a TIL population of any preceding aspect, wherein the TILs are grown in a hypoxic oxygen environment (i.e., 0-19.99% oxygen) in the second pre-REP culture. In one aspect, the oxygen percentage of the hypoxic oxygen environment is 5% O₂.

Also disclosed herein are methods of expanding tumor infiltrating lymphocytes (TILs) and/or increasing the number of Trm in a TIL population of any preceding aspect, wherein the TILs are grown in a normoxic oxygen environment (i.e., 20-22% oxygen) in the REP culture.

In one aspect, disclosed herein are methods of expanding tumor infiltrating lymphocytes (TILs) and/or increasing the number of Trm in a TIL population of any preceding aspect, wherein the TILs are grown in a hypoxic oxygen environment (i.e., 0-19.99% oxygen) in the REP culture. In one aspect, the oxygen percentage of the hypoxic oxygen environment is 5% O₂.

Also disclosed herein are methods of expanding tumor infiltrating lymphocytes (TILs) and/or increasing the number of Trm in a TIL population of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

In one aspect, disclosed herein are methods of expanding tumor infiltrating lymphocytes (TILs) and/or increasing the number of Trm in a TIL population of any preceding aspect, wherein the first pre-REP culture lasts for at least 1, 2, 3, 4, 5, 6, 7, or 8 weeks.

Also disclosed herein are methods of expanding tumor infiltrating lymphocytes (TILs) and/or increasing the number of Trm in a TIL population of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

In one aspect, disclosed herein are methods of expanding tumor infiltrating lymphocytes (TILs) and/or increasing the number of Trm in a TIL population of any preceding aspect, wherein the REP culture lasts for at least 1, 2, 3, 4, 5, 6, 7 (i.e., 1 week), 8, 9, 10, 11, 12, 13, 14 days (i.e., 2 weeks).

Also disclosed are methods of treating, inhibiting, decreasing, reducing, ameliorating, and/or preventing a cancer and/or metastasis (such as, for example, head and neck cancer and/or kidney cancer including, but not limited to renal cell carcinoma) in a subject, the method comprising a) obtaining a population of TILs (such as, for example, primary TILs obtained from an autologous, allogeneic, or syngeneic donor source); b) culturing the TILs in a first pre-rapid expansion protocol (pre-REP) culture comprising media comprising IL-2 and incubating the TILs in a normoxic oxygen environment (i.e., 20-22% oxygen); c) culturing the TILs in a second pre-REP culture comprising IL-2 and incubating the TILs in a normoxic (i.e., 20-22% oxygen) or hypoxic (i.e., 0-19.99% oxygen) oxygen environment; d) expanding TILs using a rapid expansion protocol (REP) in the presence of anti-CD3 antibody, IL-2, and allogeneic feeder cells incubating the TILs in a normoxic (i.e., 20-22% oxygen) or hypoxic (i.e., 0-19.99% oxygen) oxygen environment; and e) administering to the subject the expanded TILs of step d.

In one aspect disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a normoxic (i.e., 20-22% oxygen) oxygen environment in the second pre-REP culture.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a hypoxic (i.e., 0-19.99% oxygen) oxygen environment in the second pre-REP culture. In one aspect, the oxygen percentage of the hypoxic oxygen environment is 5% O₂.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a normoxic (i.e., 20-22% oxygen) oxygen environment in the REP culture.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a hypoxic (i.e., 0-19.99% oxygen) oxygen environment in the REP culture. In one aspect, the oxygen percentage of the hypoxic oxygen environment is 5% O₂.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the first pre-REP culture lasts for at least 1, 2, 3, 4, 5, 6, 7, or 8 weeks.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the REP culture lasts for at least 1, 2, 3, 4, 5, 6, 7 (i.e., 1 week), 8, 9, 10, 11, 12, 13, 14 days (i.e., 2 weeks).

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are obtained from an autologous donor source.

Also disclosed herein are murine models for adoptive cell therapy using TILs comprising a) obtaining a population of murine TILs (such as, for example, primary TILs obtained from an autologous, allogeneic, or syngeneic donor source); b) fragmenting the TILs; c) culturing the TILs in a first pre-rapid expansion protocol (pre-REP) culture comprising media comprising IL-2 and incubating the TILs in a normoxic (i.e., 20-22% oxygen) oxygen environment; d) culturing the TILs in a second pre-REP culture comprising IL-2 and incubating the TILs in a normoxic (i.e., 20-22% oxygen) or hypoxic (0-19.99% oxygen) oxygen environment; e) expanding TILs using a rapid expansion protocol (REP) in the presence of anti-CD3 antibody, IL-2, and allogeneic feeder cells incubating the TILs in a normoxic (i.e., 20-22% oxygen) or hypoxic (0-19.99% oxygen) oxygen environment; and f) administering to the subject the expanded TILs of step e.

In one aspect disclosed herein are murine models for adoptive cell therapy using TILs of any preceding aspect, wherein the TILs are grown in a normoxic (i.e., 20-22% oxygen) oxygen environment in the second pre-REP culture.

Also disclosed herein are murine models for adoptive cell therapy using TILs of any preceding aspect, wherein the TILs are grown in a hypoxic (0-19.99% O₂) oxygen environment in the second pre-REP culture. In one aspect, the oxygen percentage of the hypoxic oxygen environment is 5% O₂.

In one aspect disclosed herein are murine models for adoptive cell therapy using TILs of any preceding aspect, wherein the TILs are grown in a normoxic (i.e., 20-22% O₂) oxygen environment in the REP culture.

Also disclosed herein are murine models for adoptive cell therapy using TILs of any preceding aspect, wherein the TILs are grown in a hypoxic (0-19.99% O₂) oxygen environment in the REP culture. In one aspect, the oxygen percentage of the hypoxic oxygen environment is 5% O₂.

In one aspect, disclosed herein are murine models for adoptive cell therapy using TILs of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

Also disclosed herein are murine models for adoptive cell therapy using TILs of any preceding aspect, wherein the first pre-REP culture lasts for at least 1, 3, 4, 5, 6, 7, or 8 weeks.

In one aspect, disclosed herein are murine models for adoptive cell therapy using TILs of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

Also disclosed herein are murine models for adoptive cell therapy using TILs of any preceding aspect, wherein the REP culture lasts for at least 1, 2, 3, 4, 5, 6, 7 (i.e., 1 week), 8, 9, 10, 11, 12, 13, 14 days (i.e., 2 weeks).

In one aspect, disclosed herein are murine models for adoptive cell therapy using TILs of any preceding aspect, wherein the TILs are obtained from an autologous donor source.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 show twelve fragments per tumor and per culturing condition were plated in a 24-well plate. To account for hetereogeneity, tumors were cut into four pieces equal in size. Three fragments per tumor quarter were plated.

FIGS. 2A and 2B show TIL expanded from 12/13 (92.3%) of the tumors (2A). TIL from fragments cultured in α4-1BB and αPD-1 showed highest proportion (33%) of tumor-reactive TIL. IFN-γ values <21 pg/ml were regarded as non-reactive. IFN-γ values >24 pg/ml were considered as reactive (2B).

FIGS. 3A and 3B show culturing fragments in different conditions didn't have an effect on proportion of fragments that showed expansion of TIL (3A). Across all culturing condition, the majority of ex vivo expanded TIL were CD8+. Culturing fragments in IL-2+α4-1BB increased the proportion of CD8+ cells (p=0.02) (3B).

FIGS. 4A and 4B show culturing condition didn't have an effect on the memory phenotype of ex vivo expanded CD8+ TIL (4A). CD8+ TIL expanded from fragments cultured in IL-2+α4-1BB displayed an increased expression of PD-1 (p=0.009), while the proportion of 4-1BB+/PD-1-CD8+ TIL was decreased (p=0.02) (4B).

FIGS. 5A and 5B show analysis with FlowSOM revealed three distinct CD8+ populations: CD137+/CD44+ (Pop0), CD137+/CD44−(Pop4) and CD137−/CD44−(Pop6). Across all culturing conditions, Population 0 was most prevalent (82.0±1.4%) and increased in TIL that were cultured in αPD-1 (89.9±1.1%; p=0.0024) or α4-1BB (89.3±1.1%; p=0.0049).

FIGS. 6A and 6B shows in vitro expansion of TIL from RCC tumors. FIG. 6A shows that for TIL culture, tumors were resected from a primary kidney tumor site and collected in sterile conditions. Tumors collected from 44 RCC patients were minced into fragments, placed in individual wells of a 24-well plate containing media with 6000 IU/mL IL-2, and cultured for four weeks. For hypoxia experiments, primary TIL were cultured either at 20% O₂, in a standard CO₂ incubator, or placed in a tri-gas hypoxic chamber and cultured at 5% or 1% O₂ for one week. FIG. 2B shows that primary TIL were further expanded using a rapid expansion protocol (REP) by stimulation with anti-CD3 antibody in the presence of allogenic feeder cells. REP of TIL was carried out at either 20% or 5% O₂ levels. At the end of the REP process, T-cell phenotypes and reactivity to the autologous tumor of the post-REP TIL were assessed.

FIGS. 7A, 7B, 7C, and 7D characterization of TIL expanded from RCC tumors. FIG. 7A show classification of RCC tumors collected by histological subtype. FIG. 7B shows the proportion of tumors with successful TIL expansion and the percentage of tumor reactive TIL. FIG. 7C shows the average number of TIL expanded from all tumors was 71.2×10⁶ cells. FIG. 7D shows the phenotype of TIL was examined by flow cytometry. RCC TIL show primarily CD3+ T-cells, with higher proportion of CD4⁺ T-cells than CD8⁺ T-cells (p=0.04).

FIGS. 8A and 8B show culturing primary TIL in hypoxic conditions. FIG. 8A shows TIL cultured in hypoxic conditions (5% or 1% O₂) for 7 days show reduced expansion likely due to reduced proliferation. FIG. 8Bs shows CD8⁺ gated T-cells were divided based on CD45RA and CCR7 expression into four subsets: effector memory re-expressing CD45RA (EMRA), effector memory (EM), naïve or stem cell memory (N/scm), and central memory (CM). Hypoxic TIL show increased percentage of central memory-like T-cells (CD45RA⁻CCR7⁺) when compared to TIL cultured under normoxic conditions (20% O₂).

FIGS. 9A and 9B show that hypoxia leads to the formation of Tᵣₘ-like (CD69⁺CD103⁺) CD8⁺ T-cells. Tᵣₘ-like phenotype was evaluated by measuring expression of CD69 and CD103 on CD8⁺ T-cells in pre-REP TIL as well as in post-REP TIL that have been expanded in normoxia (20% O₂) or in hypoxia (5% O₂). Flow cytometry revealed an increase in the percentage of CD69⁺CD103⁺CD8⁺ TILs expanded in hypoxia as compared to those expanded in normoxia (9A), and this phenotype is observed across multiple samples FIG. 9B shows non-parametric t-test (unpaired) was used for statistical analysis; **p<0.01.

FIG. 10 shows that cytokine release is enhanced in hypoxic TIL. 20% O₂ TIL or 5% O₂ TIL were cultured in TIL complete media (TIL CM) alone, co-cultured with the autologous tumor, or cultured with autologous tumor cells treated with the W6/32 to block MHC class I. The release of IFNγ, TNFα and Granzyme B was then assessed by ELLA. When co-cultured with the autologous tumor, hypoxic TIL (5% TIL) displayed increased IFNγ, TNFα as well as increased Granzyme B release when compared to TIL expanded in normoxia (20% TIL).

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10”as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Models for Adoptive Cell Therapy (ACT)

Adoptive cell therapy (ACT) using tumor-infiltrating lymphocytes (TIL) has greatly improved treatment options for cancer patients. The success of ACT with TIL is highly dependent on the expansion of high numbers of tumor-reactive TIL.

Renal cell carcinoma (RCC) is the most common form of urinary tumors with estimated incidence of 80,000 cases per year in the U.S. RCC is classified based on histologic features into two main subtypes: clear cell RCC, as well as variant histologies such as papillary and chromophobe RCC. Clear cell is the most common subtype and accounts for 70% of the cases, while papillary and chromophobe RCC account for 10-15% and 5%, respectively.

Recent advances in immunotherapy checkpoint inhibitors have greatly improved survival rates. However, many patients develop resistance to these therapies and the overall prognosis remains poor.

Establishing a mouse model that resembles TIL therapy in human will allow us to more efficiently explore additional strategies to improve TIL therapy. Accordingly, disclosed herein are murine models for adoptive cell therapy using TILs comprising a) obtaining a population of murine TILs (such as, for example, primary TILs obtained from an autologous, allogeneic, or syngeneic donor source); b) fragmenting the TILs; c) culturing the TILs in a first pre-rapid expansion protocol (pre-REP) culture comprising media comprising IL-2 and incubating the TILs in a normoxic (i.e., 20-22% O₂) oxygen environment; d) culturing the TILs in a second pre-REP culture comprising IL-2 and incubating the TILs in a normoxic (i.e., 20-22% O₂) or hypoxic (0-19.99% O₂) oxygen environment; e) expanding TILs using a rapid expansion protocol (REP) in the presence of anti-CD3 antibody, IL-2, and allogeneic feeder cells incubating the TILs in a normoxic (i.e., 20-22% (2) or hypoxic (0-19.99% O₂) oxygen environment; and f) administering to the subject the expanded TILs of step e.

The disclosed culturing methods can comprise the use of normoxic or hypoxic oxygen environments. As used herein, “normoxic” refers to O₂ percentages between 20 and 22$ oxygen including, but not limited to 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, or 22.0% O₂. “Hypoxic” refers to O₂ percentages less than 20.0%. That is, a hypoxic oxygen environment can comprise from 0-19.99% O₂, from about 0.5% O₂ to about 10% O₂; from about 1% to about 5% O₂, from about 5% to about 10% O₂, from about 5% to about 15% O₂, from about 10% to about 15% O₂, from about 10% to about 19.99% O₂. In one aspect, the O₂ percentage is less than 19.99%, 19%, 18%, 17%, 16%, 15%, 14%, 13%, 12%, 11%, 10%, 9%, 8%, 7%, 6%, 5%, 4%, 3%, 2%, 1%, or 0.5% O₂. For example, the O₂ percentage can be 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5% O₂.

In one aspect disclosed herein are murine models for adoptive cell therapy using TILs of any preceding aspect, wherein the TILs are grown in a normoxic (i.e., 20-22% oxygen) oxygen environment in the second pre-REP culture.

Also disclosed herein are murine models for adoptive cell therapy using TILs of any preceding aspect, wherein the TILs are grown in a hypoxic (0-19.99% O₂) oxygen environment in the second pre-REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

In one aspect disclosed herein are murine models for adoptive cell therapy using TILs of any preceding aspect, wherein the TILs are grown in a normoxic (i.e., 20-22% O₂) oxygen environment in the REP culture.

Also disclosed herein are murine models for adoptive cell therapy using TILs of any preceding aspect, wherein the TILs are grown in a hypoxic (0-19.99% O₂) oxygen environment in the REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

In one aspect, disclosed herein are murine models for adoptive cell therapy using TILs of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

Also disclosed herein are murine models for adoptive cell therapy using TILs of any preceding aspect, wherein the first pre-REP culture lasts for at least 1, 3, 4, 5, 6, 7, or 8 weeks.

In one aspect, disclosed herein are murine models for adoptive cell therapy using TILs of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

Also disclosed herein are murine models for adoptive cell therapy using TILs of any preceding aspect, wherein the REP culture lasts for at least 1, 2, 3, 4, 5, 6, 7 (i.e., 1 week), 8, 9, 10, 11, 12, 13, 14 days (i.e., 2 weeks).

In one aspect, disclosed herein are murine models for adoptive cell therapy using TILs of any preceding aspect, wherein the TILs are obtained from an autologous donor source.

### C. −+Method of Expanding Tumor Infiltrating Lymphocytes and Treating Cancer

The tumor microenvironment (TME) of RCC is characterized by being highly hypoxic. This hypoxic TME can be a major factor in resistance to immunotherapy agents and poor prognosis. Novel strategies in overcoming this resistance is pivotal in advancing therapeutic approaches and improving the overall patient survival. Cellular adoptive cell transfer (ACT) with tumor infiltrating lymphocytes (TIL) has shown pronounced success in melanoma patients. This approach may form a promising strategy in RCC. Herein we show that tumor reactive TIL can be generated successfully from primary RCC tumors. We further show an advantage of adapting TIL to hypoxic conditions in improving their overall anti-tumor response.

In one aspect, disclosed herein are methods of expanding tumor infiltrating lymphocytes (TILs) and/or increasing the number of Trm in a TIL population comprising a) obtaining a population of TILs (such as, for example, primary TILs obtained from an autologous, allogeneic, or syngeneic donor source); b) culturing the TILs in a first pre-rapid expansion protocol (pre-REP) culture comprising media comprising IL-2 and incubating the TILs in a normoxic oxygen environment (i.e., 20-22% (2); c) culturing the TILs in a second pre-REP culture comprising IL-2 and incubating the TILs in a normoxic (i.e., 20-22% O₂) or hypoxic oxygen environment (i.e., 0-19.99% O₂); and d) expanding TILs using a rapid expansion protocol (REP) in the presence of anti-CD3 antibody, IL-2, and allogeneic feeder cells incubating the TILs in a normoxic (i.e., 20-22% O₂) or hypoxic (i.e., 0-19.99% O₂) oxygen environment.

Also disclosed herein are methods of expanding tumor infiltrating lymphocytes (TILs) and/or increasing the number of Trm in a TIL population of any preceding aspect, wherein the TILs are grown in a normoxic oxygen environment (i.e., 20-22% O₂) in the second pre-REP culture.

In one aspect, disclosed herein are methods of expanding tumor infiltrating lymphocytes (TILs) and/or increasing the number of Trm in a TIL population of any preceding aspect, wherein the TILs are grown in a hypoxic oxygen environment (i.e., 0-19.99% O₂) in the second pre-REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

Also disclosed herein are methods of expanding tumor infiltrating lymphocytes (TILs) and/or increasing the number of Trm in a TIL population of any preceding aspect, wherein the TILs are grown in a normoxic oxygen environment (i.e., 20-22% O₂) in the REP culture.

In one aspect, disclosed herein are methods of expanding tumor infiltrating lymphocytes (TILs) and/or increasing the number of Trm in a TIL population of any preceding aspect, wherein the TILs are grown in a hypoxic oxygen environment (i.e., 0-19.99% O₂) in the REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

Also disclosed herein are methods of expanding tumor infiltrating lymphocytes (TILs) and/or increasing the number of Trm in a TIL population of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

In one aspect, disclosed herein are methods of expanding tumor infiltrating lymphocytes (TILs) and/or increasing the number of Trm in a TIL population of any preceding aspect, wherein the first pre-REP culture lasts for at least 1, 2, 3, 4, 5, 6, 7, or 8 weeks.

Also disclosed herein are methods of expanding tumor infiltrating lymphocytes (TILs) and/or increasing the number of Trm in a TIL population of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

In one aspect, disclosed herein are methods of expanding tumor infiltrating lymphocytes (TILs) and/or increasing the number of Trm in a TIL population of any preceding aspect, wherein the REP culture lasts for at least 1, 2, 3, 4, 5, 6, 7 (i.e., 1 week), 8, 9, 10, 11, 12, 13, 14 days (i.e., 2 weeks).

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer (including, but not limited to renal cell carcinoma (RCC)); lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

Also disclosed are methods of treating, inhibiting, decreasing, reducing, ameliorating, and/or preventing a cancer and/or metastasis (such as, for example, head and neck cancer and/or kidney cancer including, but not limited to renal cell carcinoma) in a subject, the method comprising a) obtaining a population of TILs (such as, for example, primary TILs obtained from an autologous, allogeneic, or syngeneic donor source); b) culturing the TILs in a first pre-rapid expansion protocol (pre-REP) culture comprising media comprising IL-2 and incubating the TILs in a normoxic oxygen environment (i.e., 20-22% O₂); c) culturing the TILs in a second pre-REP culture comprising IL-2 and incubating the TILs in a normoxic (i.e., 20-22% O₂) or hypoxic (i.e., 0-19.99% O₂) oxygen environment; d) expanding TILs using a rapid expansion protocol (REP) in the presence of anti-CD3 antibody, IL-2, and allogeneic feeder cells incubating the TILs in a normoxic (i.e., 20-22% O₂) or hypoxic (i.e., 0-19.99% O₂) oxygen environment; and e) administering to the subject the expanded TILs of step d.

In one aspect disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a normoxic (i.e., 20-22% O₂) oxygen environment in the second pre-REP culture.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a hypoxic (i.e., 0-19.99% O₂) oxygen environment in the second pre-REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a normoxic (i.e., 20-22% O₂) oxygen environment in the REP culture.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are grown in a hypoxic (i.e., 0-19.99% O₂) oxygen environment in the REP culture. In one aspect, the oxygen percentage of the hypoxic oxygen environment is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0% O₂.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the pre-REP and/or REP cultures further comprise an anti-PD1 antibody, an anti-CTLA4 antibody, and anti-TIM-3 antibody, an anti-CD40 antibody, or an anti-4-1BB antibody.

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the first pre-REP culture lasts for at least 1, 2, 3, 4, 5, 6, 7, or 8 weeks.

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the second pre-REP culture lasts for at least 1, 2, 3, 4, 5, 6, 7 (i.e., 1 week), 8, 9, 10, 11, 12, 13, 14 days (i.e., 2 weeks).

Also disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the REP culture lasts for at least 1, 2, 3, 4, 5, 6, 7 (i.e., 1 week), 8, 9, 10, 11, 12, 13, 14 days (i.e., 2 weeks).

In one aspect, disclosed herein are methods of treating, inhibiting, decreasing, reducing, ameliorating, and/or preventing a cancer and/or metastasis (such as, for example, head and neck cancer and/or kidney cancer including, but not limited to renal cell carcinoma) of any preceding aspect, wherein the TILs are obtained from an autologous donor source.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Ex Vivo Expansion of Tumor-Infiltrating Lymphocytes (TIL) from Murine Head and Neck Tumors

Adoptive cell therapy (ACT) using tumor-infiltrating lymphocytes (TIL) has greatly improved treatment options for melanoma patients. The success of ACT with TIL is highly dependent on the expansion of high numbers of tumor-reactive TIL. Establishing a mouse model that resembles TIL therapy in human will allow us to more efficiently explore additional strategies to improve TIL therapy.

### a) Methods

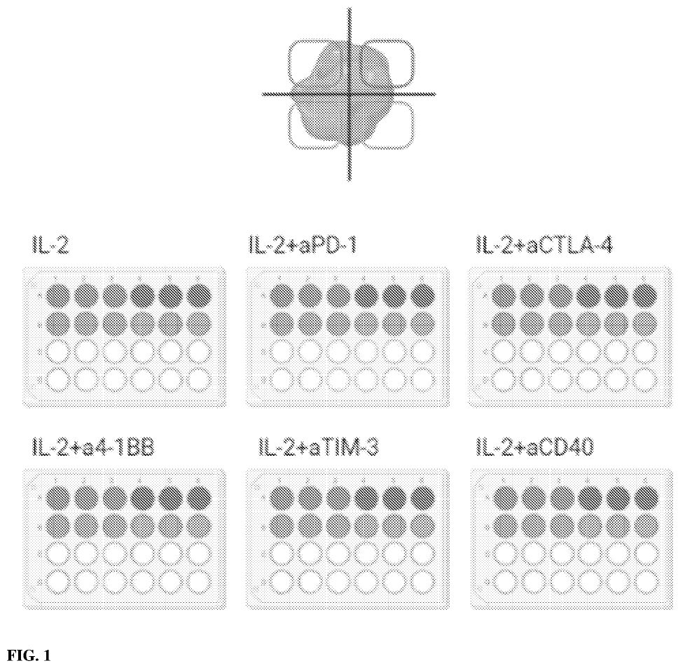

HPV+ MEER tumors were established in C57BL/6 mice. Mouse tumors were then collected, minced into 0.5-1 mm² fragments and placed in media containing 6000 IU/ml IL-2 (FIG. 1). Additionally, we compared different culturing conditions by adding αPD-1, αTIM-3, α4-1BB, αCD40 and αCTLA-4. Tumor reactivity was determined by co-culturing TIL with the tumor cell line MEER and measuring IFN-γ with ELLA after 48 hours. In addition to conventional flow gating, we used FlowSOM applying nine clusters to further analyze the impact of different ex vivo culturing conditions on the distribution of distinct cell populations.

### b) Results

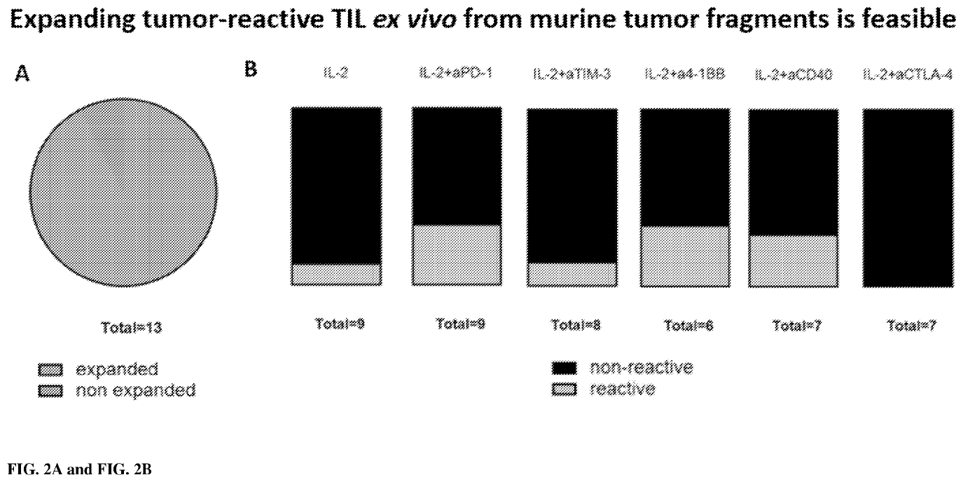

To determine if expanding tumor reactive TIL ex vivo from murine tumor fragments was feasible, TILs were expanded from 12 out of 13 (92.3%) of the tumors (FIG. 2A) and differing culture conditions compared (IL-2; IL-2 and anti-PD1 antibody; IL-2 and anti-TIM3 antibody; IL-2 and 4-1BB, IL-2 and anti-CD40; and IL-2 and anti-CTLA-4 antibody). TIL from fragments cultured in α4-1BB and αPD-1 showed highest proportion (33%) of tumor-reactive TIL with IFN-γ values <21 pg/ml were regarded as non-reactive. IFN-γ values >24 pg/ml were considered as reactive (FIG. 2B).

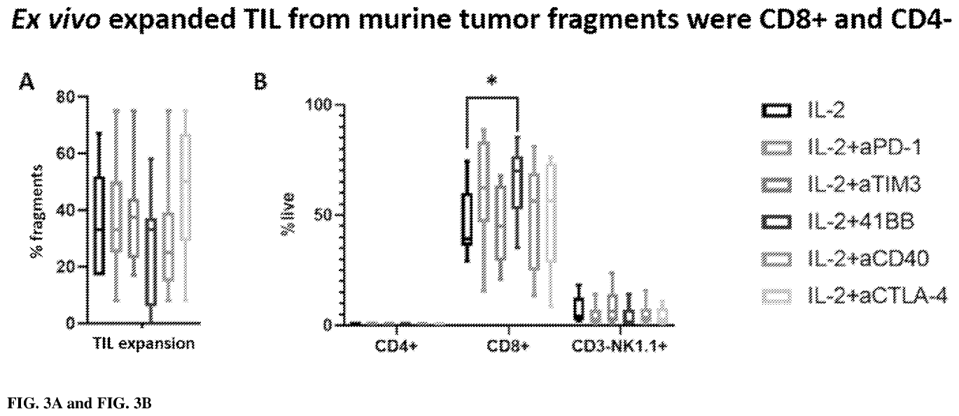

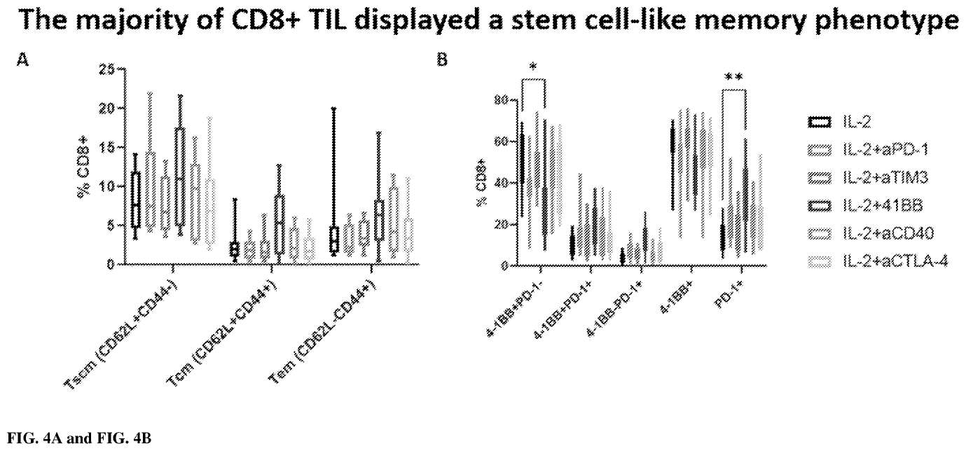

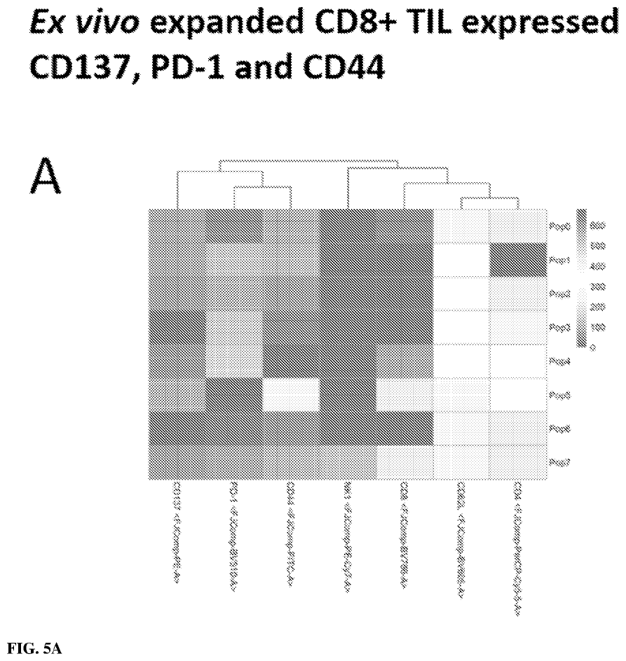

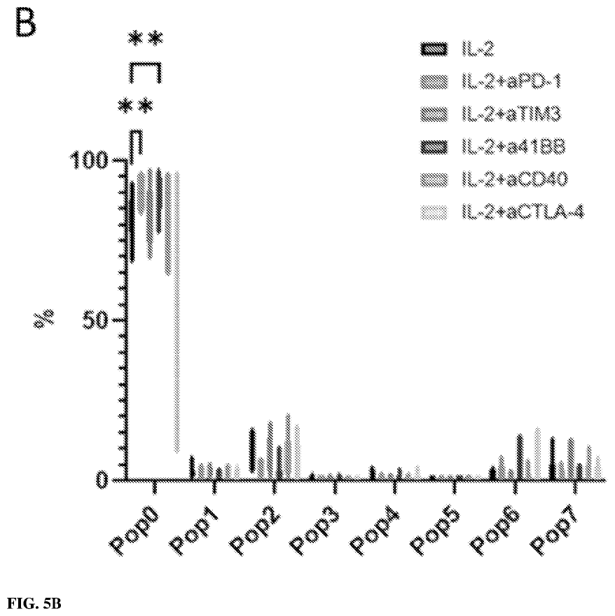

TILs from murine tumor fragments were expanded ex vivo in each culture condition and measured for total expansion (FIG. 3A) and percentage of CD8+, CD4+ and NK1.1+ cells. The results showed that ex vivo expanded TIL from murine tumor fragments were predominantly CD8+ and CD4− (FIG. 3B). We further investigated the phenotype of the Cd8+ T cells using CD62L and CD44 as markers. We found that the majority of CD8+ TIL displayed a stem cell-like memory phenotype and culturing condition didn't have an effect on the memory phenotype of ex vivo expanded CD8+ TIL (FIG. 4A). We further found that CD8+ TIL expanded from fragments cultured in IL-2+α4-1BB displayed an increased expression of PD-1 (p=0.009), while the proportion of 4-1BB+/PD-1-CD8+ TIL was decreased (FIG. 4B). Lastly we measured each culture condition and found that ex vivo expanded CD8+ TL expressed CD137, PD-1, and CD44 (FIG. 5A and 5B).

### c) Discussion

The feasibility of ex vivo expanding tumor-reactive TIL from murine tumor fragments is an outstanding resource that will enable us to examine new strategies to improve TIL therapy. It is the first milestone to generate a complete model for ACT with TIL in mouse. This will allow for not only testing different conditions during ex vivo TIL expansion, but also for testing methods to precondition the tumor prior to tumor resection as well as testing combinational therapies during ACT.

### 2. Example 2: Ex Vivo Expansion of Tumor-Infiltrating Lymphocytes (TIL) from Renal Cell Carcinoma (RCC) Tumors

Renal cell carcinoma (RCC) is the most common form of urinary tumors with estimated incidence of 80,000 cases per year in the U.S. RCC is classified based on histologic features into two main subtypes: clear cell RCC, as well as variant histologies such as papillary and chromophobe RCC. Clear cell is the most common subtype and accounts for 70% of the cases, while papillary and chromophobe RCC account for 10-15% and 5%, respectively. Recent advances in immunotherapy checkpoint inhibitors have greatly improved survival rates. However, many patients develop resistance to these therapies and the overall prognosis remains poor. The tumor microenvironment (TME) of RCC is characterized by being highly hypoxic. This hypoxic TME can be a major factor in resistance to immunotherapy agents and poor prognosis. Novel strategies in overcoming this resistance is pivotal in advancing therapeutic approaches and improving the overall patient survival. Cellular adoptive cell transfer (ACT) with tumor infiltrating lymphocytes (TIL) has shown pronounced success in melanoma patients. This approach may form a promising strategy in RCC. In this study, we aim to show that tumor reactive TIL can be generated successfully from primary RCC tumors. We further show an advantage of adapting TIL to hypoxic conditions in improving their overall anti-tumor response.

### a) Methods And Results

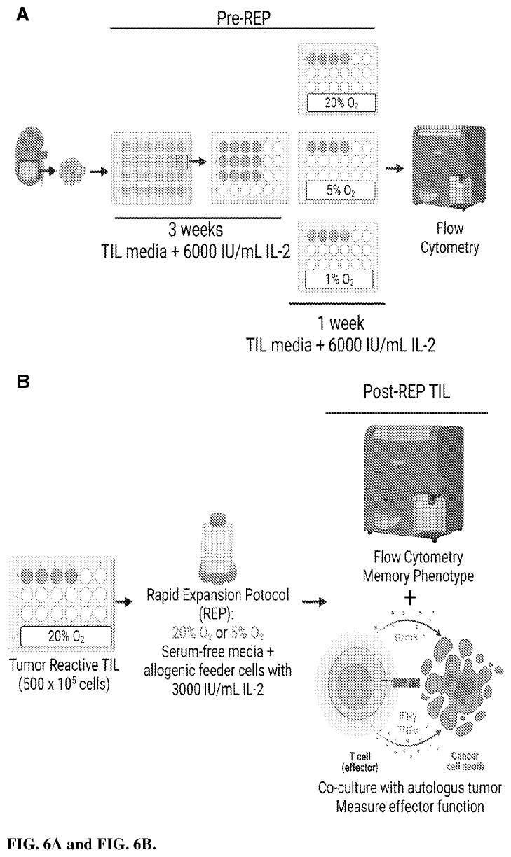

To investigate if manipulations of invitro expansion of TILs could have an impact on efficacy and tumor resistance, tumors were resected from a primary kidney tumor site and collected in sterile conditions. Tumors collected from 44 RCC patients were minced into fragments, placed in individual wells of a 24-well plate containing media with 6000 IU/mL IL-2, and cultured for four weeks. For hypoxia experiments, primary TIL were cultured either at 20% O₂, in a standard CO₂ incubator, or placed in a tri-gas hypoxic chamber and cultured at 5% or 1% O₂ for one week (FIG. 6A). Primary TIL were further expanded using a rapid expansion protocol (REP) by stimulation with anti-CD3 antibody in the presence of allogenic feeder cells. REP of TIL was carried out at either 20% or 5% O₂ levels. At the end of the REP process, T-cell phenotypes and reactivity to the autologous tumor of the post-REP TIL were assessed (FIG. 6B).

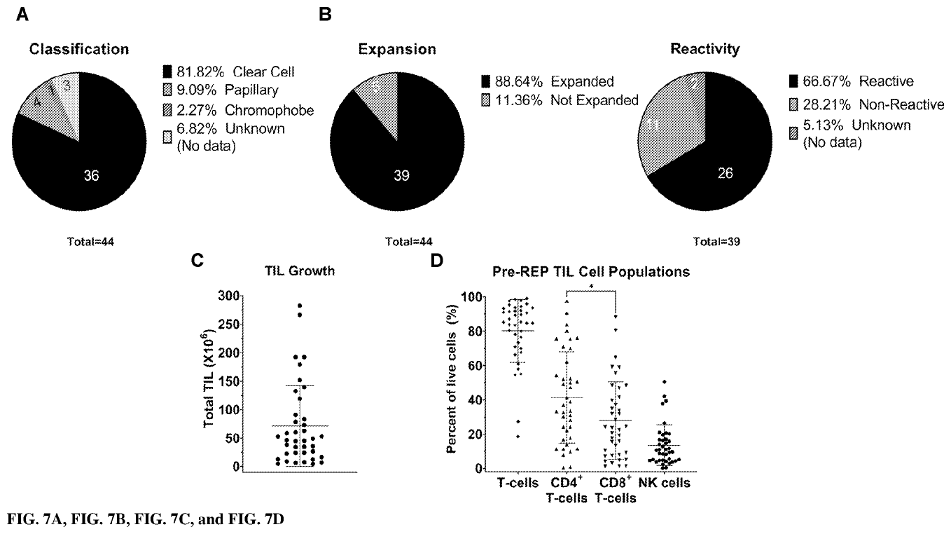

Next, we wanted to show that tumor reactive TIL can be successfully expanded from RCC tumors. To do this, tumors were first classified based on histological subtype (FIG. 7A). Next we characterized the expansion and reactivity of TILs (FIG. 7B), the number of expanded TILs (FIG. 7B) and calculated the number of lymphocytes by total T cells, CD4+ T cells, CD8+ T cells, or natural killer (NK) cells (FIG. 7D).

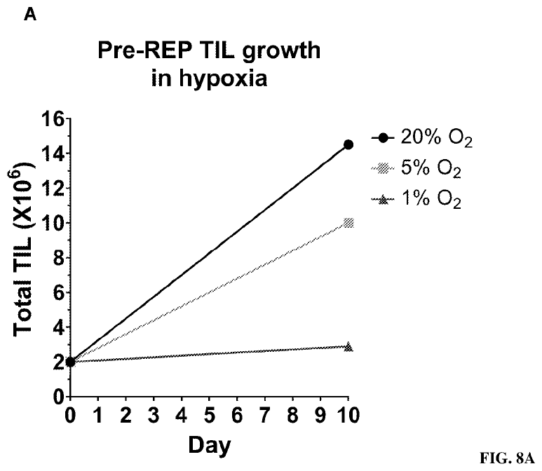

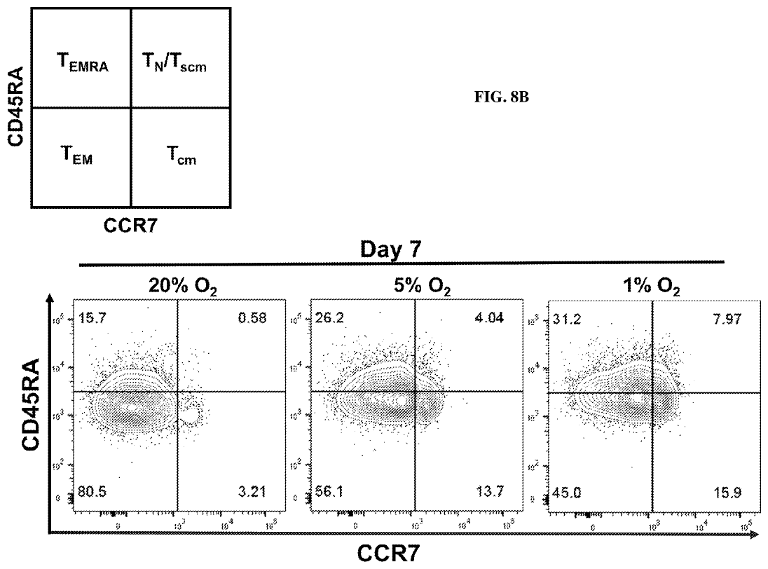

To determine the effect of the oxygen percentage during culture on the TIL population, TILs were cultured in normal (20% O₂ or hypoxic conditions (5% or 1% O₂) for 7 days. TILs cultured in hypoxic conditions show reduced expansion likely due to reduced proliferation (FIG. 8A). CD8⁺ gated T-cells were divided based on CD45RA and CCR7 expression into four subsets: effector memory re-expressing CD45RA (EMRA), effector memory (EM), naïve or stem cell memory (N/scm), and central memory (CM). Hypoxic TIL show increased percentage of central memory-like T-cells (CD45RA⁻CCR7⁺) when compared to TIL cultured under normoxic conditions (20% O₂) (FIG. 8B). Together the date shows that primary TIL cultured in hypoxia display a memory-like phenotype.

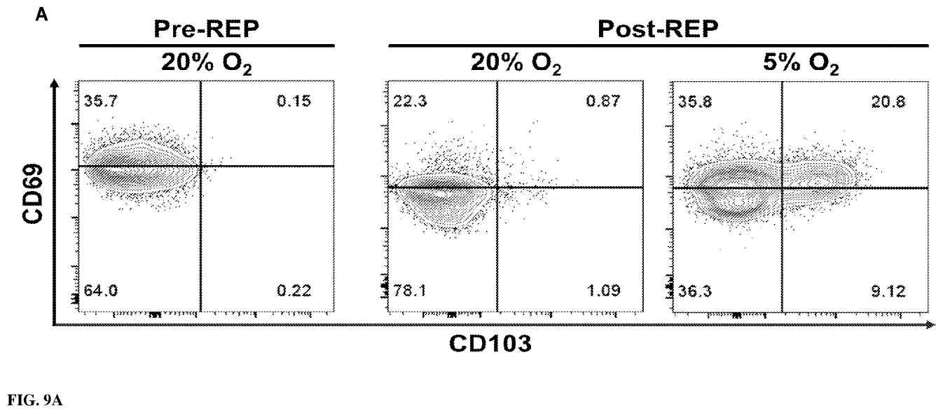

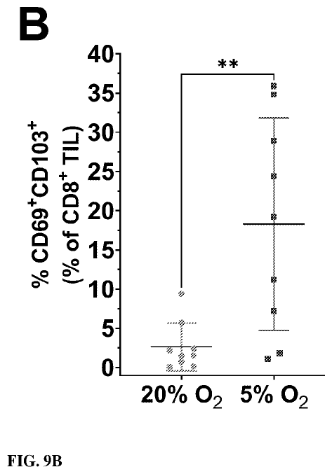

Due to the memory-like phenotype of TILs, we next expanded TILs in hypoxic conditions to see the effect on T cells to form resident memory Trm T cells. Trm-like phenotype was evaluated by measuring expression of CD69 and CD103 on CD8⁺ T-cells in pre-REP TIL as well as in post-REP TIL that have been expanded in normoxia (20% O₂) or in hypoxia (5% O₂). Flow cytometry revealed an increase in the percentage of CD69⁺CD103⁺CD8⁺ TILs expanded in hypoxia as compared to those expanded in normoxia, and this phenotype is observed across multiple samples (FIG. 9A and 9B). Together the date show that expanded hypoxic TIL display a tissue resident memory T-cell (Tᵣₘ)-like phenotype.

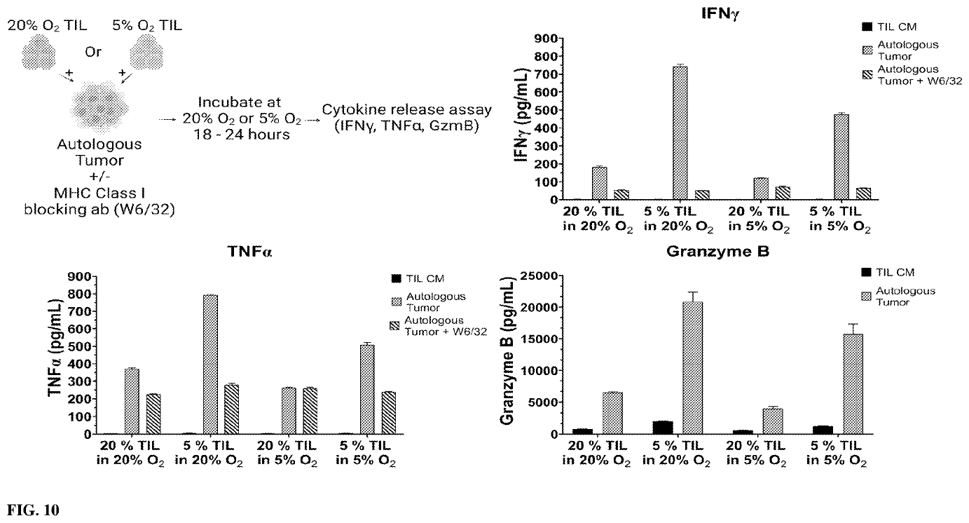

Examining effector functions of the hypoxic TIL howed enhanced effector function in response to autologous tumor (FIG. 10). Specifically, when co-cultured with the autologous tumor, hypoxic TIL (5% TIL) displayed increased IFNγ, TNFα as well as increased Granzyme B release when compared to TIL expanded in normoxia (20% TIL).

### b) Discussion

Herein we show that functional tumor reactive TIL can be expanded from RCC tumors. Additionally, we show that hypoxia induces a memory like-phenotype in RCC TIL. Moreover, adapting TIL to hypoxic conditions can be effective in producing TIL with enhanced anti-tumor reactivity.

1. A method of expanding tumor infiltrating lymphocytes (TILs) comprising
a. obtaining a population of TILs;
b. culturing the TILs in a first pre-rapid expansion protocol (pre-REP) culture comprising media comprising IL-2 and incubating the TILs in a normoxic oxygen environment;
c. culturing the TILs in a second pre-REP culture comprising IL-2 and incubating the TILs in a normoxic or hypoxic oxygen environment; and
d. expanding TILs using a rapid expansion protocol (REP) in the presence of anti-CD3 antibody, IL-2, and allogeneic feeder cells incubating the TILs in a normoxic or hypoxic oxygen environment.

7. (canceled).

8. The method of claim 1, wherein the pre-REP and/or REP cultures further comprise an anti-PD1 antibody, an anti-CTLA4 antibody, and anti-TIM-3 antibody, an anti-CD40 antibody, or an anti-4-1BB antibody.

9. The method of claim 1, wherein the first pre-REP culture lasts for 3 weeks.

10. The method of claim 1, wherein the second pre-REP culture lasts for 1 week.

11. The method of claim 1, wherein the REP culture lasts for 1 week.

12. A method of increasing the number of Trm in a TIL population comprising
e. obtaining a population of TILs;
f. culturing the TILs in a first pre-rapid expansion protocol (pre-REP) culture comprising media comprising IL-2 and incubating the TILs in a normoxic oxygen environment;
g. culturing the TILs in a second pre-REP culture comprising IL-2 and incubating the TILs in a normoxic or hypoxic oxygen environment; and
h. expanding TILs using a rapid expansion protocol (REP) in the presence of anti-CD3 antibody, IL-2, and allogeneic feeder cells incubating the TILs in a normoxic or hypoxic oxygen environment.

13-18. (canceled).

19. The method of claim 12, wherein the pre-REP and/or REP cultures further comprise an anti-PD1 antibody, an anti-CTLA4 antibody, and anti-TIM-3 antibody, an anti-CD40 antibody, or an anti-4-1BB antibody.

20. The method of claim 12, wherein the first pre-REP culture lasts for 3 weeks.

21. The method of claim 12, wherein the second pre-REP culture lasts for 1 week.

22. The method of claim 12, wherein the REP culture lasts for 1 week.

23. A method of treating a cancer in a subject, the method comprising
i. obtaining a population of TILs;
j. culturing the TILs in a first pre-rapid expansion protocol (pre-REP) culture comprising media comprising IL-2 and incubating the TILs in a normoxic oxygen environment;
k. culturing the TILs in a second pre-REP culture comprising IL-2 and incubating the TILs in a normoxic or hypoxic oxygen environment;
l. expanding TILs using a rapid expansion protocol (REP) in the presence of anti-CD3 antibody, IL-2, and allogeneic feeder cells incubating the TILs in a normoxic or hypoxic oxygen environment; and
m. administering to the subject the expanded TILs of step d.

24-29. (canceled).

30. The method of claim 23, wherein the pre-REP and/or REP cultures further comprise an anti-PD1 antibody, an anti-CTLA4 antibody, and anti-TIM-3 antibody, an anti-CD40 antibody, or an anti-4-1BB antibody.

31. The method of claim 23, wherein the first pre-REP culture lasts for 3 weeks.

32. The method of claim 23, wherein the second pre-REP culture lasts for 1 week.

33. The method of claim 23, wherein the REP culture lasts for 1 week.

34. The method of claim 23, wherein the TILs are obtained from an autologous donor source.

35. A murine model for adoptive cell therapy using TILs comprising
n. obtaining a population of murine TILs;
o. fragmenting the TILs;
p. culturing the TILs in a first pre-rapid expansion protocol (pre-REP) culture comprising media comprising IL-2 and incubating the TILs in a normoxic oxygen environment;
q. culturing the TILs in a second pre-REP culture comprising IL-2 and incubating the TILs in a normoxic or hypoxic oxygen environment;
r. expanding TILs using a rapid expansion protocol (REP) in the presence of anti-CD3 antibody, IL-2, and allogeneic feeder cells incubating the TILs in a normoxic or hypoxic oxygen environment; and
s. administering to the subject the expanded TILs of step e.

36-41. (canceled).

42. The method of claim 35, wherein the pre-REP and/or REP cultures further comprise an anti-PD1 antibody, an anti-CTLA4 antibody, and anti-TIM-3 antibody, an anti-CD40 antibody, or an anti-4-1BB antibody.

43. The method of claim 35, wherein the first pre-REP culture lasts for 3 weeks.

44. The method of claim 35, wherein the second pre-REP culture lasts for 1 week.

45. The method of claim 35, wherein the REP culture lasts for 1 week.

46. The method of claim 35, wherein the TILs are obtained from an autologous donor source.
